(19) United States
(12) Patent Application Publication
(10) Pub. No.: US 2003/0138390 A1
(43) Pub. Date: Jul. 24, 2003

(54) Use of alcohol dehydrogenase inhibitors for cosmetic treatment of keratinous materials

(76) Inventor: Didier Saint-Leger, Courbevoie (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/257,473

(22) PCT Filed: Apr. 6, 2001
(86) PCT No.: PCT/FR01/01054

(30) Foreign Application Priority Data
Apr. 12, 2000 (FR) 00/04709

(51) Int. Cl. A61K 7/06

(52) U.S. Cl. 424/70.1

(57) ABSTRACT

The invention concerns the use of an alcohol dehydrogenase enzyme inhibitor in cosmetic treatment of keratinous materials, to prevent, attenuate and/or eliminate undesirable effects of primary alcohols at the surface layers of the skin and its appendages.

The present invention relates to the use of agents that inhibit the enzyme alcohol dehydrogenase in the cosmetic treatment of keratin materials, to prevent, alleviate and/or suppress the adverse effects of primary alcohols on the superficial layers of the skin and of its appendices.

The use of these agents that inhibit the enzyme alcohol dehydrogenase also makes it possible to combat the premature ageing of skin tissues caused by primary alcohols.

Primary alcohols of general structure R—CH₂OH, like ethanol, for example, are agents commonly used in cosmetic products. In most cases, they are used as carriers acting as solvents, but they can also be used for their specific properties, like glycerol, for example, which is used to moisturize the skin.

It has been found that primary alcohols impair the structures and functions of the superficial skin layers, these modifications not necessarily being detectable to the naked eye. In the long term, these impairments result in drying or rapid desquamation on the skin surface.

In particular, impairments of the capillary circulation, capable of resulting in redness and rashes, have been observed.

It has been established that these skin complaints are caused by aldehydes formed following the enzymatic catalysis of primary alcohols by the enzyme alcohol dehydrogenase (Jonathan K. Wilkin, M.D. Journal of Investigation Dermatology, 1980, V. 91, No. 2, pages 117-119).

The effects of the enzyme alcohol dehydrogenase have also been studied in order to treat ethylene glycol ingestions or toxicities caused by ethanol.

Moreover, the inhibitors of the enzyme alcohol dehydrogenase are used in biochemical research or with a therapeutic aim, in order to combat alcohol poisoning (Biochemical and Biophysical Research Communication, V. 203, No. 3, 1994, 30 September 1994).

The Applicant has discovered that primary alcohols also have a negative action on the tissues of the superficial layer of the epidermis, which predisposes these tissues to various types of attack by, for example, surfactant detergents, and that the use of inhibitors of the enzyme alcohol dehydrogenase in cosmetic compositions containing a primary alcohol can prevent, alleviate and/or suppress the adverse effects of primary alcohols on the superficial layers of the skin and of its appendices.

One subject of the invention is, in particular, the use of an inhibitor of the enzyme alcohol dehydrogenase, in order to combat the premature ageing of skin tissues caused by the harmful effects of primary alcohols.

Another subject of the present invention is a process for the cosmetic treatment of keratin materials.

The present invention also relates to an agent for the cosmetic treatment of keratin materials.

Another subject of the invention is a cosmetic composition comprising at least one inhibitor of the enzyme alcohol dehydrogenase, in a cosmetically acceptable medium containing at least one primary alcohol.

Other subjects of the invention will become apparent in the light of the following description and examples.

The invention relates to the use of an inhibitor of the enzyme alcohol dehydrogenase, preferably human, in the cosmetic treatment of keratin materials, to prevent, alleviate and/or suppress the adverse effects of primary alcohols on the superficial layers of the skin and of its appendices. The expression “superficial layer of the skin and of its appendices” means the stratum corneum, the epidermis, the scalp, the hair follicles, the sebaceous glands, the mucous membranes and the eccrine or apocrine sweat glands.

The inhibitors of the enzyme alcohol dehydrogenase may especially be used to combat the premature ageing of skin tissues caused by the adverse effects of primary alcohols.

The inhibitors of the enzyme alcohol dehydrogenase can be present in a concentration from 0.0001 to 50% by weight, preferably from 0.01 to 10% by weight and even more preferably from 0.1 to 10%, relative to the total weight of the composition.

The inhibitors of the enzyme alcohol dehydrogenase may be chosen from, without being limited to, auramine O, allicin, 1,5-anilinonaphthalenesulfonic acid, 1,7-anilinonaphthalenesulfonic acid, 1,8-anilinonaphthalenesulfonic acid, berberine, canavanine, 2,2′-diprypyl, imidazole, m-methylbenzamide, 4-methylpyrazole, pyrazole, 4-pentylpyrazole, O-phenanthroline, alrestatin, anthranic acid, O-carboxybenzaldehyde, 2,3-dimethylsuccinic acid, ethacrynic acid, isonicotinic acid, phenacamide, quercetin, quercitrin, sorbinil, tetramethyleneglutaric acid, valproic acid, propranolol, 2,2,2-trichloroethanol, 4,5-diaminopyrazole and its derivatives and 2-ethyl-5-methyl-2H-3,4-diaminopyrazole.

According to one preferred embodiment of the invention, an inhibitor resulting in a percentage of inhibition in vitro of at least 50%, assessed using a Sigma Diagnostics Kit (reference 333-B), with an ethanol concentration of 5.75 10⁻⁵ M and an inhibitor concentration of 10⁻³ M, is used. Among the preferred inhibitors, mention may be made of pyrazole, 4-methylpyrazole, imidazole, 2,2,2-trichloroethanol, 4,5-diaminopyrazole and its derivatives substituted on the amine functions and/or on the pyrazole ring, and 2-ethyl-5-methyl-2H-3,4-diaminopyrazole.

The inhibitors of the enzyme alcohol dehydrogenase defined above may be used in compositions which may be applied simultaneously, concomitantly or sequentially before or after with a cosmetic composition containing at least one primary alcohol.

Preferably, they are applied simultaneously or concomitantly.

The primary alcohols have a general structure R—CH₂OH, in which R is a linear or branched, saturated or unsaturated, cyclic or noncyclic, hydrocarbon-based chain, which may include one or more hetero atoms and one or more other hydroxyl groups. Preferably, the hetero atoms are chosen from sulfur, oxygen and nitrogen atoms. Preferably, R denotes a linear or branched alkyl radical containing 1 to 30 carbon atoms and preferably 1 to 15 carbon atoms.

Alcohols which may be used according to the invention are ethanol, n-propanol, n-butanol, polyethylene glycols, 1,3-propanediol, dodecanol, cetyl alcohol, oleyl alcohol, lauryl alcohol, stearyl alcohol, myristyl alcohol, behenyl alcohol, linoleyl alcohol, glycerol and 1,2-propanediol, these last two compounds containing both primary and secondary alcohol functions.

The cosmetic compositions according to the invention may be found in various forms, for example as lotions, milks, creams, shampoos, sprays, mousses, sticks, etc.

The cosmetic compositions of the invention may further comprise, depending on their intended applications, additives other than the primary alcohols defined above, and chosen from fatty substances, organic solvents, gelling agents, emollients, softeners, antioxidants, opacifiers, stabilizers, silicones, antifoams, anti-hydrating agents, vitamins, fragrances, preservatives, anionic, cationic, nonionic, amphoteric or zwitterionic surfactants, or mixtures thereof, fillers, sequestering agents, polymers, propellants, basifying or acidifying agents, colorants, pigments, thickeners, free-radical scavengers, sunscreens or any other additive commonly used in cosmetics.

The cosmetic composition may also be used as a cosmetic agent in the form of a kit. These cosmetic agents comprise at least two components:

a first component comprising at least one primary alcohol and

a second component comprising at least one inhibitor of the enzyme alcohol dehydrogenase,

these two components being intended to be applied simultaneously, concomitantly or sequentially on the skin tissues to be protected.

The present invention also relates to a process for the cosmetic treatment of keratin materials consisting in the application of at least two components:

a first component comprising at least one primary alcohol and

a second component comprising at least one inhibitor of the enzyme alcohol dehydrogenase,

these two components being intended to be applied simultaneously, concomitantly or sequentially on the skin tissues to be protected.

The present invention also relates to a cosmetic composition comprising, in a cosmetically acceptable medium, at least one primary alcohol as defined above, and at least one inhibitor of the enzyme alcohol dehydrogenase resulting, in vitro, in an at least 50% inhibition of the activity of the alcohol dehydrogenase.

The compositions may contain cosmetic additives, such as those mentioned above.

The following examples are intended to illustrate the invention without, however, being limiting.

### EXAMPLES OF FORMULATIONS

| | |
|---|---|
| Lotion for preventing hair loss | |
| Ethanol | 30% |
| 2, 2, 2-Trichloroethanol | 5% |
| 2,4-Diaminopyrimidine 3-N-oxide | 1.5% |
| Fragrance | 0.5% |
| Water | qs 100 |
| Sun gel with alcohol | |
| Ethanol | 8% |
| Imidazole | 3% |
| Carbopol 980 | 0.3% |
| Triethanolamine | 0.4% |
| Methyl para-hydroxybenzoate | 0.2% |
| Water | qs 100 |
| Shampoo with alcohol | |
| Sodium lauryl ether sulfate | 16% |
| Tegobetaine | 8% |
| Ethanol | 5% |
| 4-Methylpyrazole | 1% |
| Polyethylene glycol dioleate | 3% |
| Citric acid | 2.3% |
| Sodium chloride | 0.9% |
| para-Hydroxybenzoic acid esters | 0.5% |
| Water | qs 100 |
| Moisturizing cream | |
| Cetylstearyl alcohol and cetylstearyl | 7% |
| alcohol polyethylene glycol ether | |
| (CTFA name: cetearyl alcohol and | |
| ceteareth 30) sold under the name | |
| Nonidac 1618F by the company | |
| Condea | |
| Glyceryl mono-distearate sold under | 2% |
| the name Geleol pastilles by the | |
| company Gattefosse | |
| Cetyl alcohol sold under the name | 1.5% |
| Lanette 16 NF by the company Cognis | |
| Silicone microemulsion sold under the | 1.5% |
| name DC200 Fluid 350 cs by the | |
| company Dow Corning | |
| Liquid petroleum jelly sold under the | 15% |
| name Marcol 82 by the company Esso | |
| Butyl p-hydroxybenzoate | 0.2% |
| Pure glycerol codex, synthetic | 20% |
| or plant-based | |
| Pyrazole | 4% |
| Imidazolidinylurea | 0.2% |
| Distilled water | qs 100 |

Test of Inhibition of the Activity of the Enzyme Alcohol Dehydrogenase

The inhibition of the activity of the enzyme alcohol dehydrogenase was determined using a Sigma Diagnostics kit (ref. 333-B).

3 ml of the Alcohol Reagent, 10 μl of ethanol solution (0.08% ethanol w/v ref. 330-20) and 10 μl of inhibitor solution in distilled water are incubated for 10 minutes at room temperature.

For each assay, a blank consisting of 3 ml of the Alcohol Reagent, 10 μl of distilled water and 10 μl of inhibitor solution in distilled water is prepared.

The inhibitors are diluted so as to be at a concentration of 10⁻³ M in the final mixture.

After incubation of the mixture, 100 μl are taken up and read over a 10-minute interval at 340 nm. The readings are performed in 96-well UV microplates using a UV-visible multiwell spectrophotometer (Spectramax Plus—Molecular Devices) against the blank.

The inhibition results are expressed as a percentage of absorbance at 340 nm relative to a reference (no inhibitor in the mixture):

| Inhibitor | % |
|---|---|
| Reference | 100 |
| Pyrazole | 16 |
| 4-Methylpyrazole | 35 |
| Imidazole | 32 |
| 2, 2, 2-Trichloroethanol | -12 |
| 2-Ethyl-5-methyl-2H-3,4-diaminopyracole | 31 |

1. The use of an inhibitor of the enzyme alcohol dehydrogenase in the cosmetic treatment of keratin materials, to prevent, alleviate and/or suppress the adverse effects of primary alcohols on the superficial layers of the skin and of its appendices.

2. The use as claimed in claim 1, characterized in that the superficial layers of the skin and of its appendices are the stratum corneum, the epidermis, the scalp, the hair follicles, the sebaceous glands, the mucous membranes and the eccrine or apocrine sweat glands.

3. The use as claimed in claim 1 or 2, with the aim of combating the premature ageing of skin tissues caused by the adverse effects of primary alcohols.

4. The use as claimed in any one of claims 1 to 3, characterized in that the compound that is an inhibitor of the enzyme alcohol dehydrogenase is present in a concentration from 0.0001 to 50% by weight, relative to the total weight of the composition.

5. The use as claimed in any one of claims 1 to 4, characterized in that the compound that is an inhibitor of the enzyme alcohol dehydrogenase is present in a concentration from 0.01 to 10% by weight and preferably from 0.1% to 10% by weight, relative to the total weight of the composition.

6. The use as claimed in any one of claims 1 to 5, characterized in that the compound which is an inhibitor of the enzyme alcohol dehydrogenase is chosen from auramine O, allicin, 1,5-anilinonaphthalenesulfonic acid, 1,7-anilinonaphthalenesulfonic acid, 1,8-anilinonaphthalenesulfonic acid, berberine, canavanine, 2,2′-diprypyl, imidazole, m-methylbenzamide, pyrazole, 4-methylpyrazole, 4-pentylpyrazole, O-phenanthroline, alrestatin, anthranic acid, O-carboxybenzaldehyde, 2,3-dimethylsuccinic acid, ethacrynic acid, isonicotinic acid, phenacamide, quercetin, quercitrin, sorbinil, tetramethyleneglutaric acid, valproic acid, propranolol, 2,2,2-trichloroethanol, 4,5-diaminopyrazole and its derivatives and 2-ethyl-5-methyl-2H-3,4-diaminopyrazole.

7. The use as claimed in any one of claims 1 to 6, characterized in that the compound that is an inhibitor of the enzyme alcohol dehydrogenase results in a percentage of inhibition in vitro of at least 50%.

8. The use as claimed in claim 7 of an inhibitor chosen from pyrazole, 4-methylpyrazole, imidazole, 2,2,2-trichloroethanol, 4,5-diaminopyrazole and its derivatives substituted on the amine functions and/or on the pyrazole ring, and 2-ethyl-5-methyl-2H-3,4-diaminopyrazole.

9. The use as claimed in any one of claims 1 to 8, characterized in that the inhibitors of the enzyme alcohol dehydrogenase are applied on the keratin materials simultaneously, concomitantly or sequentially with a cosmetic composition containing at least one primary alcohol.

10. The use as claimed in claim 9, characterized in that the primary alcohol corresponds to the formula R—CH₂OH, in which R is a linear or branched, saturated or unsaturated, cyclic or noncyclic, hydrocarbon-based chain, which may include one or more hetero atoms and one or more other hydroxyl groups.

11. The use as claimed in claim 10, characterized in that the radical R denotes a linear or branched alkyl radical containing 1 to 30 carbon atoms.

12. The use as claimed in either of claims 10 or 11, characterized in that the hetero atoms are chosen from sulfur, oxygen and nitrogen atoms.

13. The use as claimed in any one of claims 9 to 12, characterized in that the primary alcohol is chosen from ethanol, n-propanol, n-butanol, polyethylene glycols, 1,3-propanediol, dodecanol, cetyl alcohol, oleyl alcohol, lauryl alcohol, stearyl alcohol, myristyl alcohol, behenyl alcohol, linoleyl alcohol, glycerol and 1,2-propanediol.

14. A process for the cosmetic treatment of keratin materials, characterized in that it consists of the application of at least two components:
a first component comprising at least one primary alcohol and
a second component comprising at least one inhibitor of alcohol dehydrogenase,
these two components being applied simultaneously, concomitantly or sequentially on the skin tissues to be protected.

15. A cosmetic agent in the form of a kit, comprising at least two components:
a first component comprising at least one primary alcohol and
a second component comprising at least one inhibitor of alcohol dehydrogenase,
these two components being intended to be applied simultaneously, concomitantly or sequentially on the skin tissues to be protected.

16. A cosmetic composition comprising, in a cosmetically acceptable medium, at least one primary alcohol, and at least one inhibitor resulting, in vitro, in an at least 50% inhibition of the activity of alcohol dehydrogenase.

17. The composition as claimed in claim 16, containing as an inhibitor of alcohol dehydrogenase, a compound chosen from pyrazole, 4-methylpyrazole, imidazole, 2,2,2-trichloroethanol, 4,5-diaminopyrazole and its derivatives substituted on the amine functions and/or on the pyrazole ring, and 2-ethyl-5-methyl-2H-3,4-diaminopyrazole.

18. The composition as claimed in either of claims 16 or 17, characterized in that it also contains at least one additive other than the primary alcohols defined above, chosen from fatty substances, organic solvents, gelling agents, emollients, softeners, antioxidants, opacifiers, stabilizers, silicones, antifoams, anti-hydrating agents, vitamins, fragrances, preservatives, anionic, cationic, nonionic, amphoteric or zwitterionic surfactants, or mixtures thereof, fillers, sequestering agents, polymers, propellants, basifying or acidifying agents, colorants, pigments, thickeners, free-radicals scavengers and sunscreens.
